(19) United States
(12) Patent Application Publication
(10) Pub. No.: US 2002/0132765 A1
(43) Pub. Date: Sep. 19, 2002

(54) Streptogramin derivatives, their preparation and compositions containing them

(76) Inventors: Eric Bacque, Gif Sur Yvette (FR); Jean-Claude Barriere, Gometz Le Chatel (FR); Gerard Puchault, Marcilly (FR)

Correspondence Address: Finnegan, Henderson, Farabow,, Garrett & Dunner, L.L.P., 1300 I Street, N.W., Washington, DC, 20005-3315, US

(21) Appl. No.: 10/055,888

(22) Filed: Jan. 28, 2002

Related U.S. Application Data
(63) Continuation of application No. PCT/FR00/02147, filed on Jul. 26, 2000.
(60) Provisional application No. 60/152,268, filed on Sep. 3, 1999.

(30) Foreign Application Priority Data
Jul. 27, 1999 (FR) 99 09709

(51) Int. Cl. A61K 38/12; C07K 11/02

(52) U.S. Cl. 514/9; 530/317

(57) ABSTRACT

Group B streptogramin derivatives of formula (I) and salts thereof:

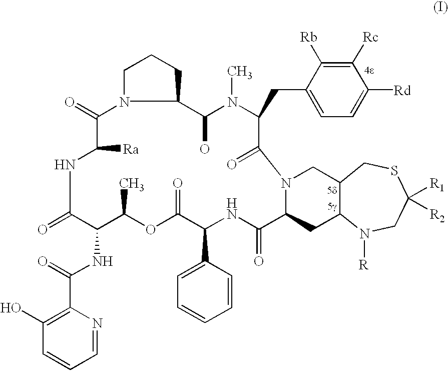

alone or in combination with at least one group A streptogramin derivative or salt thereof, pharmaceutical compositions comprising at least one streptogramin chosen from group B streptogramin derivatives of formula (I) and salts thereof, alone or in combination with at least one streptogramin chosen from group A streptogramin derivatives and salts thereof, and processes of preparing at least one streptogramin chosen from group B streptogramin derivatives of formula (I) and salts thereof.

This application is a continuation application under 35 U.S.C. §111(a) of International patent application no. PCT/FR00/02147, filed Jul. 26, 2000, and claims priority under 35 U.S.C. §119 to French patent application no. 99/09709, filed Jul. 27, 1999, and U.S. provisional patent application No. 60/152,268, filed Sep. 3, 1999.

The present invention relates to group B streptogramin derivatives of formula

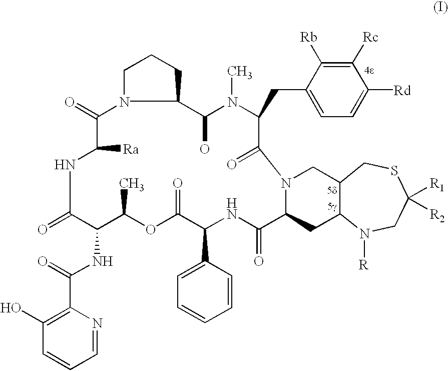

and salts thereof, when they exist, which have advantageous antibacterial activity, alone or in combination with at least one group A streptogramin derivative.

Among the known streptogramins, pristinamycin, an antibacterial agent of natural origin produced by Streptomyces pristinaespiralis, was isolated for the first time in 1955. The pristinamycin sold under the name Pyostacine® comprises mainly pristinamycin IA combined with pristinamycin IIA.

Another antibacterial agent of the streptogramin family, virginiamycin, was isolated from Streptomyces virginiae, ATCC 13161 [Antibiotics and Chemotherapy, 5, 632 (1955)]. Virginiamycin (Staphylomycine®) comprises mainly factor S combined with factor M₁.

Group B streptogramin derivatives, for example, have been described in European patents and patent applications EP 133 097, EP 248 703, EP 770 132, and EP 772 630. Such group B streptogramin derivatives include, for example, semisynthetic streptogramin derivatives of formula (A), and salts thereof:

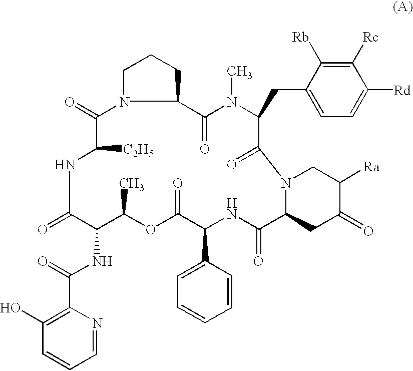

wherein:

(A) —Ra is chosen from

(1) —CH₂R′a groups, wherein R′a is an unsubstituted or substituted heterocyclylthio group, and

(2) ═CHR′a groups, wherein R′a is chosen from

(a) substituted alkylamino groups,

(b) substituted alkyloxy groups,

(c) substituted alkylthio groups,

(d) unsubstituted or substituted heterocyclylamino groups,

(e) unsubstituted or substituted heterocyclyloxy groups, and

(f) unsubstituted or substituted heterocyclylthio groups,

—Rb is a hydrogen atom,

—Rc is a hydrogen atom, and

—Rd is chosen from a hydrogen atom and a dimethylamino group, or alternatively

(B) —Ra is a hydrogen atom,

—Rb is chosen from a hydrogen atom and a methyl group, and

—Rc and Rd are chosen from a hydrogen atom and known substituents.

When combined with a semisynthetic group A streptogramin derivative, these derivatives show synergistic action and can be used as antibacterial agents either via injection alone or via the oral route alone.

The inventors have now found that group B streptogramin derivatives of formula (I) and salts thereof:

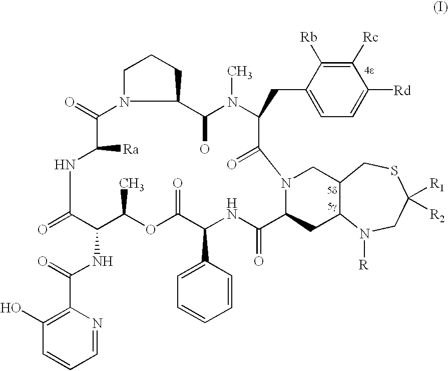

wherein:

R is chosen from a hydrogen atom, a methyl group, alkyl groups of formula R′—CH₂—, wherein R′ is chosen from straight and branched alkyl groups, and acyl groups unsubstituted or substituted with hydroxyl,

R₁ and R₂, which are identical or different, are each chosen from a hydrogen atom and alkyl groups,

Ra is chosen from a methyl group and an ethyl group, and

Rb, Rc and Rd are defined as follows:

1) Rb and Rc are each a hydrogen atom, and Rd is chosen from a hydrogen atom, a methylamino group, and a dimethylamino group, or

2) Rb is a hydrogen atom, Rc is chosen from a hydrogen atom, a chlorine atom, a bromine atom, and (C₃-C₅) alkenyl groups, and Rd is chosen from —N(CH₃)-R′″ groups, wherein R′″ is chosen from:

(a) alkyl groups,

(b) C₂-C₄ hydroxyalkyl groups,

(c) unsubstituted C₂-C₈ alkenyl groups,

(d) C₂-C₈ alkenyl groups substituted with (i) an unsubstituted or substituted phenyl group, (ii) an unsubstituted or substituted cycloalkyl(C₃-C₆)methyl group, (iii) an unsubstituted benzyl group, (iv) a benzyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups, or (v) heterocyclylmethyl groups and heterocyclylethyl groups, wherein the heterocyclyl portions of the heterocyclylmethyl groups and the heterocyclylethyl groups are chosen from saturated and unsaturated 5-or 6-membered heterocyclyl groups comprising from 1 or 2 heteroatoms chosen from a sulphur atom, an oxygen atom, and a nitrogen atom, and wherein the heterocyclyl groups are unsubstituted or substituted with a group chosen from alkyl groups, C₂-C₈ alkenyl groups, C₃-C₆ cycloalkyl groups, saturated and unsaturated 4- to 6-membered heterocyclyl groups, an unsubstituted phenyl group, a benzyl group, or a phenyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups,

(e) a cyanomethyl group,

(f) a carboxymethyl group, and

(g) —CORe groups and —CH₂CORe groups, wherein Re is chosen from (i) —OR′e groups, wherein R′e is chosen from C₁-C₆ alkyl groups, C₂-C₆ alkenyl groups, a benzyl group, and heterocyclylmethyl groups, wherein the heterocyclyl portion is chosen from 5- or 6- membered heterocyclyl groups comprising from 1 or 2 heteroatoms chosen from a sulphur atom, an oxygen atom, and a nitrogen atom, (ii) alkylamino groups, (iii) alkylmethylamino groups, (iv) heterocyclylamino groups and heterocyclylmethylamino groups, wherein the heterocyclyl portion of the heterocyclylamino groups and the heterocyclylmethylamino groups is chosen from 5- or 6-membered saturated heterocyclyl groups comprising from 1 or 2 heteroatoms chosen from a sulphur atom, an oxygen atom, and a nitrogen atom, and wherein the heterocyclyl groups are unsubstituted or substituted with a group chosen from alkyl groups, a benzyl group, and alkyloxycarbonyl groups, or

3) Rb is a hydrogen atom, Rd is chosen from an —NHCH₃ group and an —N(CH₃)₂ group, and Rc is chosen from a chlorine atom and a bromine atom, and when Rd is an —N(CH₃)₂ group, Rc is chosen from C₃-C₅ alkenyl groups, or

4) Rb and Rd are each a hydrogen atom, and Rc is chosen from halogen atoms, alkylamino groups, dialkylamino groups, alkyloxy groups, a trifluoromethoxy group, thioalkyl groups, C₁-C₆ alkyl groups, and trihalomethyl groups, or

5) Rb and Rc are hydrogen atoms, and Rd is chosen from halogen atoms, an ethylamino group, a diethylamino group, a methylethylamino group, alkyloxy groups, a trifluoromethoxy group, alkylthio groups, alkylsulfinyl groups, alkylsulfonyl groups, alkyl (1 to 6C) groups, a phenyl group, and trihalomethyl groups, or

6) Rb is a hydrogen atom, Rc is chosen from halogen atoms, alkylamino groups, dialkylamino groups, alkyloxy groups, a trifluoromethoxy group, thioalkyl groups, and C₁-C₃ alkyl groups, and Rd is chosen from halogen atoms, an amino group, alkylamino groups, dialkylamino groups, alkyloxy groups, a trifluoromethoxy group, thioalkyl groups, C₁-C₆ alkyl groups, and trihalomethyl groups, or

7) Rc is a hydrogen atom, and Rb and Rd are each a methyl group, show advantageous antibacterial activities, for example, both orally and parenterally.

Group B streptogramin derivatives of formula (I) are advantageous, for example, because of their powerful oral and parenteral activity, which gives them an undeniable advantage especially in the case of treating serious infections, in a hospital environment via injection, followed by an oral ambulatory treatment which is easier to administer to the patients. Thus, the practitioner is not obliged to change the patient's category of medicinal product between the end of the hospital treatment and the overall end of the treatment.

In one embodiment of the invention, for example, in formula (I) above, the halogen atoms can be chosen from a fluorine atom, a chlorine atom, a bromine atom, and an iodine atom. Further, for example, the alkyl groups and the acyl groups are straight or branched and, except where particularly mentioned, contain from 1 to 4 carbon atoms.

Moreover, the stereochemistry of the ring at 5γ,5δ may be 5γ(R),5δ(S) or 5γ(S),5δ(R). It is understood that the compounds of the form 5γ(R),5δ(S) and 5γ(S),5δ(R) and also mixtures thereof fall within the context of the present invention.

According to the invention, streptogramin derivatives of formula (I), or salts thereof, may be prepared, for example, by

A) reacting an amino mercaptan of formula (II):

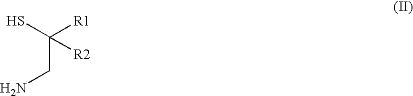

wherein

R₁ and R₂, which are identical or different, may each be chosen from a hydrogen atom and alkyl groups,

with a streptogramin derivative of formula (III):

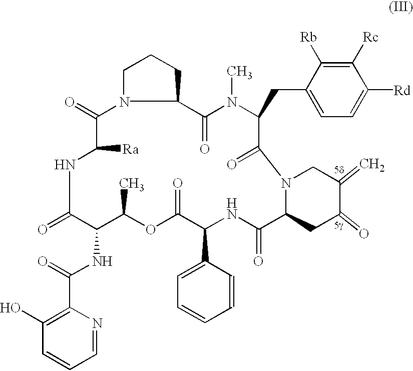

wherein

Ra is chosen from a methyl group and an ethyl group, and

Rb, Rc and Rd are defined as follows:

1) Rb and Rc are each a hydrogen atom and Rd is chosen from a hydrogen atom, a methylamino group, and a dimethylamino group, or

2) Rb is a hydrogen atom, Rc is chosen from a hydrogen atom, a chlorine atom, a bromine atom, and (C₃-C₅) alkenyl groups, and Rd is chosen from —N(CH₃)—R′″ groups, wherein R′″ is chosen from

(a) alkyl groups,

(b) C₂-C₄ hydroxyalkyl groups,

(c) unsubstituted C₂-C₈ alkenyl groups,

(d) C₂-C₈ alkenyl groups substituted with (i) an unsubstituted or substituted phenyl group, (ii) an unsubstituted or substituted cycloalkyl(C₃-C₆)methyl group, (iii) an unsubstituted benzyl group, (iv) a benzyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups, or (v) heterocyclylmethyl groups and heterocyclylethyl groups, wherein the heterocyclyl portions of the heterocyclylmethyl groups and the heterocyclylethyl groups are chosen from saturated and unsaturated 5-or 6-membered heterocyclyl groups comprising from 1 or 2 heteroatoms chosen from a sulphur atom, an oxygen atom, and a nitrogen atom, and wherein the heterocyclyl groups may be unsubstituted or substituted with a group chosen from alkyl groups, C₂-C₈ alkenyl groups, C₃-C₆ cycloalkyl groups, saturated and unsaturated 4- to 6-membered heterocyclyl groups, an unsubstituted phenyl group, a benzyl group, or a phenyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups,

(e) a cyanomethyl group,

(f) a carboxymethyl group, and

(g) —CORe groups and —CH₂CORe groups, wherein Re is chosen from (i) —OR′e groups, wherein R′e is chosen from C₁-C₆ alkyl groups, C₂-C₆ alkenyl groups, a benzyl group, and heterocyclylmethyl groups, wherein the heterocyclyl portion is chosen from 5- or 6-membered heterocyclyl groups comprising from 1 or 2 heteroatoms chosen from a sulphur atom, an oxygen atom, and a nitrogen atom, (ii) alkylamino groups, (iii) alkylmethylamino groups, (iv) heterocyclylamino groups and heterocyclylmethylamino groups, wherein the heterocyclyl portion of the heterocyclylamino groups and the heterocyclylmethylamino groups is chosen from 5- or 6-membered saturated heterocyclyl groups comprising from 1 or 2 heteroatoms chosen from a sulphur atom, an oxygen atom, and a nitrogen atom, and wherein the heterocyclyl groups may be unsubstituted or substituted with a group chosen from alkyl groups, a benzyl group, and alkyloxycarbonyl groups, or

3) Rb is a hydrogen atom, Rd is chosen from an —NHCH₃ group and an —N(CH₃)₂ group, and Rc is chosen from a chlorine atom and a bromine atom, and when Rd is an —N(CH₃)₂ group, Rc is chosen from C₃-C₅ alkenyl groups, or

4) Rb and Rd are each a hydrogen atom, and Rc is chosen from halogen atoms, alkylamino groups, dialkylamino groups, alkyloxy groups, a trifluoromethoxy group, thioalkyl groups, C₁-C₆ alkyl groups, and trihalomethyl groups, or

5) Rb and Rc are hydrogen atoms, and Rd is chosen from halogen atoms, an ethylamino group, a diethylamino group, a methylethylamino group, alkyloxy groups, a trifluoromethoxy group, alkylthio groups, alkylsulfinyl groups, alkylsulfonyl groups, C₁-C₆ alkyl groups, a phenyl group, and trihalomethyl groups, or

6) Rb is a hydrogen atom, Rc is chosen from halogen atoms, alkylamino groups, dialkylamino groups, alkyloxy groups, a trifluoromethoxy group, thioalkyl groups, and C₁-C₃ alkyl groups, and Rd is chosen from halogen atoms, an amino group, alkylamino groups, dialkylamino groups, alkyloxy groups, a trifluoromethoxy group, thioalkyl groups, C₁-C₆ alkyl groups, and trihalomethyl groups, or

7) Rc is a hydrogen atom, and Rb and Rd are each a methyl group, to prepare at least one 5δ-aminoethylthiomethyl derivative,

B) reducing at least one 5δ-aminoethylthiomethyl derivative prepared above in (A) to prepare at least one group B streptogramin derivative,

C) optionally separating said at least one group B streptogramin derivative,

D) optionally substituting, at the R position of formula (I), said at least one group B streptogramin derivative prepared in (B) or (C) above with an R group chosen from a hydrogen atom, a methyl group, alkyl groups of formula R′—CH₂—, wherein R′ is chosen from straight and branched alkyl groups, and acyl groups unsubstituted or substituted with a hydroxyl group, and

E) optionally converting a streptogramin derivative obtained in (B), (C), or (D) above to an acid addition salt.

Addition of amino mercaptans of formula (II) can be carried out, for example, in an organic solvent such as an alcohol, for example, methanol, or a chlorinated solvent, for example, dichloromethane, dichloroethane, or chloroform, or in a mixture of such solvents, at a temperature ranging, for example, from −30 to 60° C. Such a process can also be performed in an inert medium, for example, under nitrogen or under argon.

Reduction can be carried out, for example, according to known, art-recognized methods that do not affect the rest of the molecule. For example, the process can be performed in the presence of a hydride, for example, an alkali metal borohydride, such as sodium borohydride, or, for example, an alkali metal cyanoborohydride, such as sodium cyanoborohydride, in an organic solvent, such as a nitrile, for example, acetonitrile, in acetic medium, at a temperature ranging, for example, from −20 to 60° C. Such a process can be performed in an inert medium, for example, under nitrogen or under argon.

When R is chosen from alkyl groups as defined above, substitution with an R group can be performed, for example, by treating a corresponding derivative for which R is a hydrogen atom, in a reductive medium, with an aldehyde of formula (IV):

R—CHO (IV)

wherein R is chosen from a hydrogen atom, a methyl group, and alkyl groups of formula R′—CH₂—, wherein R′ is chosen from straight and branched alkyl groups, and acyl groups unsubstituted or substituted with a hydroxyl group.

Such a process can be performed, for example, in an organic solvent such as a nitrile, for example, acetonitrile, in acetic medium, at a temperature ranging, for example, from −20 to 60° C. Reductive conditions are implemented by any method that does not affect the rest of the molecule, such as in the presence of a hydride, for example, alkali metal borohydride, such as sodium borohydride, and, for example, alkali metal cyanoborohydride, such as sodium cyanoborohydride. Such a process can be performed, for example, in an inert medium, for example, under nitrogen or under argon.

When R is chosen from acyl groups unsubstituted or substituted with a hydroxyl group, substitution with an R group can be carried out, for example, by acylation of a derivative obtained for which R is a hydrogen atom. Acylation can be carried out, for example, by any known, art-recognized method that does not affect the rest of the molecule, for example, by treatment with a reactive acid derivative such as the acid chloride or a reactive ester under known, art-recognized conditions for adding an acid derivative to an amine, such as in the presence of a tertiary amine, for example, triethylamine, or a coupling agent, for example, carbodiimide, at a temperature ranging, for example, from 0 to 60° C., in an organic solvent such as a chlorinated solvent, for example, chloroform or dichloromethane, an amide, for example, dimethylformamide or N-methylpyrrolidone, or an ether, such as, for example, tetrahydrofuran.

When it is desired to obtain a compound for which R is chosen from acyl groups substituted with a hydroxyl group, it is possible to react an acid derivative whose hydroxyl function has been protected beforehand, or to react a corresponding haloderivative and then hydroxylate the reacted haloderivative obtained.

Protection of the hydroxyl group can be carried out, for example, with any protecting group whose installation and removal does not affect the rest of the molecule, such as according to T. W. Greene and P. G. M. Wuts, Protective Groups in Organic Synthesis (2^{nd} edition), A. Wiley-Interscience Publication (1991).

Separation of the stereoisomers can be carried out according to known, art-recognized methods, for example, by chromatography or by crystallization.

Streptogramin derivatives of formula (Ill) may be prepared according to known, art-recognized methods, such as the methods described in European patent nos. EP 133 098 and EP 432 029, or by analogy with these methods or the methods described in European patent nos. EP 248 703, EP 770 132, EP 772 630 and EP 821 697, or described below in the examples.

Streptogramin derivatives of formula (I) may be purified, where necessary, by known, art-recognized physical methods, such as crystallization or chromatography.

Some of the streptogramin derivatives of formula (I) may be converted into acid addition salts, by known, art-recognized methods. It is understood that such salts, when they exist, also fall within the context of the present invention.

Representative addition salts with pharmaceutically acceptable acids include, for example, salts formed with inorganic acids, such as hydrochlorides, hydrobromides, sulfates, nitrates, phosphates, and derivatives thereof, and salts formed with organic acids, such as succinates, fumarates, tartrates, acetates, propionates, maleates, citrates, methanesulfonates, ethanesulfonates, phenylsulfonates, p-toleunesulfonates, isethionates, naphthalenesulfonates, camphorsulfonates, and derivatives thereof.

Where appropriate, streptogramin derivatives bearing a carboxyl substituent may be converted into metal salts or into addition salts with nitrogenous bases according to known, art-recognized methods. Such salts can be obtained, for example, by reacting a metallic base, such as an alkali metal base or an alkaline-earth metal base, reacting ammonia, or reacting an amine on a streptogramin derivative according to the invention, in a suitable solvent such as an alcohol, an ether or water, or by exchange reaction with a salt of an organic acid. Such a salt precipitates after optionally concentrating the solution, and is separated out by filtration, settling or lyophilization. Non-limiting examples of pharmaceutically acceptable salts according to the invention include salts with alkali metals, such as sodium, potassium, and lithium, salts with alkaline-earth metals, such as magnesium and calcium, ammonium salts, and salts of nitrogenous bases, such as ethanolamine, diethanolamine, trimethylamine, triethylamine, methylamine, propylamine, diisopropylamine, N,N-dimethylethanolamine, benzylamine, dicyclohexylamine, N-benzyl-β-phenethylamine, N,N′-dibenzylethylenediamine, diphenylenediamine, benzhydrylamine, quinine, choline, arginine, lysine, leucine and dibenzylamine.

Streptogramin derivatives according to the present invention have antibacterial properties and synergizing properties with respect to the antibacterial activity of the group A streptogramin derivatives. They are advantageous on account of their activity, alone or combined with group A streptogramin derivatives, and on account of their activity both orally and parenterally, which opens the way to an ambulatory relay treatment without modifying the nature of the medicinal product.

When at least one streptogramin derivative according to the invention is combined with at least one group A streptogramin derivative, such derivatives can be chosen, for example, depending on whether it is desired to obtain a form for oral or parenteral administration, from natural group A streptogramin derivatives, such as pristinamycin IIA, pristinamycin IIB, pristinamycin IIC, pristinamycin IID, pristinamycin IIE, pristinamycin IIF, pristinamycin IIG, and salts thereof, from semisynthetic derivatives, and salts thereof, for example, as described in U.S. Pat. No. 4,590,004 and European patent no. EP 191 662, and from semisynthetic derivatives of formula (α), and salts thereof:

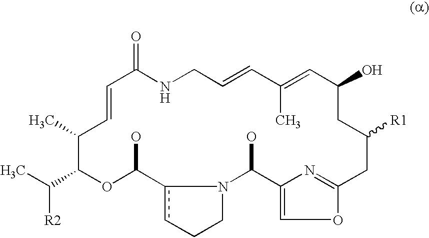

wherein:

R₁ is chosen from

(a) —NR′R″ groups, wherein R′ is chosen from a hydrogen atom and a methyl group, and R″ is chosen from a hydrogen atom, alkyl groups, cycloalkyl groups, an allyl group, a propargyl group, and a benzyl group,

(b) —OR′″ groups, wherein R′″ is chosen from a hydrogen atom, alkyl groups, cycloalkyl groups, an allyl group, a propargyl group, and a benzyl group, and

(c) —NR₃R₄ groups, wherein R₃ and R₄ are each independently a methyl group or form, together with the nitrogen atom to which they are attached, a saturated or unsaturated 4- or 5-membered heterocycle optionally comprising, in addition to the nitrogen atom, a hetero atom chosen from a nitrogen atom, an oxygen atom, and a sulfur atom,

R₂ is chosen from a hydrogen atom, a methyl group, and an ethyl group, and the bond --- is a single bond or a double bond.

Group A derivatives which may be combined therewith can also be chosen from semisynthetic derivatives of formula (β), and salts thereof:

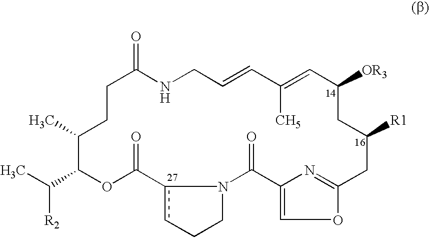

wherein:

R₁ is chosen from halogen atoms, an azido group, and a thiocyanato group,

R₂ is chosen from a hydrogen atom, a methyl group, and an ethyl group,

R₃ is chosen from a hydrogen atom and unsubstituted or substituted aliphatic ester residues, unsubstituted or substituted cycloaliphatic ester residues, unsubstituted or substituted aromatic ester residues, unsubstituted or substituted araliphatic ester residues, unsubstituted or substituted heterocyclic ester residues, and unsubstituted or substituted heterocyclylaliphatic ester residues, and the bond --- is a single bond (27R stereochemistry) or a double bond.

For example, streptogramin derivatives of formula (β) include compounds, wherein R₃ is a R′₃—CO— group, wherein R′₃ is chosen from:

(A) an unsubstituted or substituted phenyl group and unsubstituted or substituted phenylalkyl groups, wherein, when R′₃ is a substituted phenyl group or a substituted phenylalkyl group, the phenyl portion is substituted with at least one substituent chosen from

(1) alkyl groups, unsubstituted or substituted with an NR″R′″ group, wherein

(a) R″ and R′″, which are identical or different, are each chosen from a hydrogen atom and alkyl groups which can form, together with the nitrogen atom to which they are attached, a saturated or unsaturated 3- to 8-membered heterocyclyl group, optionally comprising, in addition to the nitrogen atom, another hetero atom chosen from an oxygen atom, a sulfur atom, and a nitrogen atom, wherein the heterocyclyl group is unsubstituted or substituted with at least one group chosen from saturated and unsaturated 3- to 8-membered alkyl groups, saturated and unsaturated 3- to 8-membered hydroxyalkyl groups, saturated and unsaturated 3- to 8-membered alkyloxyalkyl groups, saturated and unsaturated 3- to 8-membered alkyloxycarbonylalkyl groups, saturated and unsaturated 3- to 8-membered aryl groups, saturated and unsaturated 3- to 8-membered heterocyclyl groups, saturated and unsaturated 3- to 8-membered heterocyclylalkyl groups, and —CH₂—CO—NR″R′″ groups, wherein NR″R′″ is defined as above, or

(b) R″ and R′″, which are identical or different, are each chosen from (i) hydroxyalkyl groups, (ii) a phenyl group, and (iii) saturated and unsaturated 3- to 8-membered heterocyclylalkyl groups,

(2) alkyl groups unsubstituted or substituted with a —CO—NR″R′″ group, wherein NR″R′″ is defined as above,

(3) alkyl groups substituted with an NR″R′″ group as defined above, and

(4) acyl groups substituted with an NR″R′″ group as defined above,

(B) a substituted phenyl group and substituted phenylalkyl groups, wherein the phenyl group or phenyl portion is substituted with at least one substituent chosen from (a) alkyl groups, unsubstituted or substituted with at least one group chosen from alkyloxy groups and alkylthio groups, wherein the alkyloxy groups or alkylthio groups are unsubstituted or substituted with a carboxyl group or an NR″R′″ group as defined above, and (b) acyloxy groups which are unsubstituted or substituted with an NR″R′″ group as defined above,

(C) alkyl groups and cycloalkyl groups, wherein the alkyl groups and the cycloalkyl groups are unsubstituted or substituted with at least one group chosen from (a) a carboxyl group, (b) carboxyalkyldisulfanyl groups, (c) NR″R′″ groups, —CH₂—NR″R′″ groups, and —CO—NR″R′″ groups, wherein NR″R′″ is defined as above, (d) alkyloxycarbonyl groups, alkyloxy groups, and alkyldisulfanyl groups, wherein the alkyloxycarbonyl groups, alkyloxy groups, and alkyldisulfanyl groups are unsubstituted or substituted with an NR″R′″ group or a —CO—NR″R′″ group, wherein NR″R′″ is defined as above, and

(D) saturated and unsaturated 3- to 8-membered heterocyclyl groups, which are unsubstituted or substituted with at least one substituent chosen from alkyl groups and acyl groups, wherein the alkyl groups and the acyl groups are unsubstituted or substituted with an NR″R′″ group as defined above.

It is understood that combinations of at least one streptogramin derivative according to the invention and of at least one group A streptogramin derivative also fall within the context of the present invention.

In vitro on Staphylococcus aureus 209P, streptogramin derivatives according to the invention have been shown to be active at concentrations ranging, for example, from 0.12 μg/ml to 32 μg/ml combined with a group A streptogramin derivative, such as pristinamycin IIB, and at concentrations ranging, for example, from 0.5 μg/ml to 32 μg/ml on Staphylococcus aureus Schiclia (meticillin-resistant) combined with, for example, pristinamycin IIB. In vivo, streptogramin derivatives according to the invention synergize the antimicrobial activity of pristinamycin IIB on experimental infections of mice with Staphylococcus aureus IP8203 at doses ranging, for example, from 25 mg/kg to 150 mg/kg subcutaneously or orally (CD₅₀) [30/70 combinations].

The streptogramin derivatives according to the invention are advantageous, for example, on account of their low toxicity. None of the streptogramin derivatives according to the invention showed any toxicity at a dose of 150 mg/kg administered twice orally with a 5-hour interval.

In one embodiment, streptogramin derivatives of formula (I) and salts thereof, wherein:

R is chosen from a hydrogen atom, a methyl group, alkyl groups of formula R′—CH₂—, wherein R′ is chosen from straight and branched alkyl groups, and acyl groups unsubstituted or substituted with a hydroxyl group,

R₁ and R₂, which are identical or different, are each chosen from a hydrogen atom and alkyl groups,

Ra is an ethyl group, and

Rb, Rc, and Rd are defined as follows:

1) Rb and Rc are each a hydrogen atom, and Rd is chosen from a methylamino group and a dimethylamino group, or

2) Rb is a hydrogen atom, Rd is chosen from an —NHCH₃ group and an —N(CH₃)₂ group, and Rc is a chlorine atom,

have been found to be advantageous.

Representative streptogramin derivatives of the invention include: 5γ(S),5δ(R)[5γa,5δb]-1,4-hexahydrothiazepinopristinamycin IE, 4ε-chloro-5γ(S),5δ(R)[5γa,5δb]-1,4-hexahydrothiazepinopristinamycin IE, 5γ(R),5δ(S)-2,2-dimethyl[5γa ,5δb]-1,4-hexahydrothiazepinopristinamycin IE, and 5γ(S),5δ(R)-2,2-dimethyl-4-(4-hydroxybutyryl)[5γa ,5δb]-1,4-hexahydrothiazepinopristinamycin IE

Streptogramin derivatives of formula (α) are described in International patent application publication no. WO 99/05165.

Streptogramin derivatives of formula (β), described in French patent application no. FR 99/08375, can be prepared by halogenation, by conversion into an azide or by conversion into a thiocyanate, of a streptogramin derivative of formula (γ):

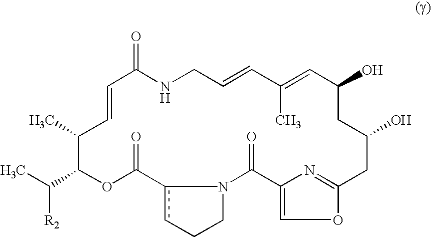

wherein R₂ is chosen from a hydrogen atom, a methyl group, and an ethyl group, the --- bond is a single bond (27R stereochemistry) or a double bond, and wherein the hydroxyl group in position 14 has been protected beforehand, followed by removal of the protecting group and, where appropriate, in order to obtain a streptogramin derivative of formula (β) for which R₃ is other than a hydrogen atom, by introduction of an aliphatic, cycloaliphatic, aromatic, araliphatic, heterocyclic or heterocyclylaliphatic ester residue which may be substituted at the R₃ position according to known, art-recognized methods which do not adversely affect the rest of the molecule.

Halogenation reactions, conversion into azides or conversion into thiocyanates can be carried out, for example, in the presence of an aminosulfur trifluoride, such as diethylaminosulfur trifluoride, bis(2-methoxyethyl)aminosulfur trifluoride (e.g., Deoxofluor®), or morpholinosulfur trifluoride, or alternatively in the presence of sulfur tetrafluoride, using a reagent such as a tetraalkylammonium (tetramethylammonium, tetraethylammonium, tetrapropylammonium or tetrabutylammonium), trialkylbenzylammonium or trialkylphenylammonium halide, azide or thiocyanate, or using an alkali metal halide, azide or thiocyanate optionally supplemented with a crown ether. The reaction can be carried out in a chlorinated organic solvent, such as dichloromethane, dichloroethane, or chloroform, or in an ether, such as tetrahydrofuran, ranging, for example, from −78° C., to 40° C., optionally under argon or nitrogen. Use of a hydroxyl derivative of (16S) configuration gives a derivative of (16R) configuration. Protection and deprotection of the hydroxyl group in position 14 can be carried out, for example, according to the known, art-recognized methods which do not adversely affect the rest of the molecule [T. W. Greene et P. G. M. Wuts, Protective Groups in Organic Synthesis (2^{nd} edition), A. Wiley—Interscience Publication (1991)].

To prepare a streptogramin derivative of formula (β) wherein R₃ is an aliphatic, cycloaliphatic, aromatic, araliphatic, heterocyclic or heterocyclylaliphatic ester which may be substituted, esterification can be carried out, for example, by reacting an acid or a reactive derivative of an acid, such as an acid chloride, reactive ester, or anhydride, in the presence or absence of a coupling agent, for example, a carbodiimide, such as dicyclohexylcarbodiimide, and a tertiary amine, for example, a trialkylamine, such as triethylamine, diisopropylethylamine, pyridine or a derivative, and optionally a catalyst such as 4-N-dimethylaminopyridine, at a temperature ranging, for example, from −40° C. to +80° C., in an organic solvent such as an amide, for example, dimethylformamide or N-methyl-2-pyrrolidinone, such as pyridine, such as a halogenated solvent, for example, dichloromethane, dichloroethane, or chloroform, or such as an ether, for example, tetrahydrofuran, dioxane, or dimethoxyethane. Functional groups which may interfere with the reaction are protected beforehand.

The examples which follow, given in a non-limiting manner, illustrate the present invention.

In the examples which follow, the NMR spectra were acquired in deuterochloroform, and the nomenclature used is that of J. O. Anteunis et al., Eur. Biochem., 58, 259 (1975), and that of the following structure, which identifies the positioning of substituents:

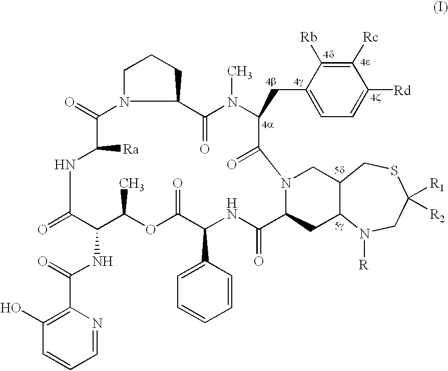

The purifications were performed by flash chromatography, using a 0.063-0.04 mm silica. As the chromatography proceeded, the fractions were analyzed by thin layer chromatography (TLC) on Merck 60F254 silica plates. The fractions corresponding to the same Rf were combined and then concentrated to dryness, under reduced pressure (30-45° C.; 2.7 kPa). The compounds thus obtained were analyzed by known, art-recognized spectroscopic techniques, such as NMR, IR, and MS, to identify the compounds obtained.

### EXAMPLE 1

### 5γ(S),5δ(R)[5γa,5δb]-1,4-hexahydrothiazepinopristinamycin IE

4 Å molecular sieves were added, under a nitrogen atmosphere, to 65 g of crude 5δ-(2-aminoethyl)thiomethylpristinamycin IA dissolved in a mixture of 1500 cm³ of acetonitrile and 150 cm³ of acetic acid. After stirring for 30 minutes at about 20° C., 5.2 g of sodium cyanoborohydride were added. The stirring was continued for 18 hours. The reaction mixture was then filtered through Clarcel® and rinsed with acetonitrile, and the filtrate was then concentrated to dryness under reduced pressure (2.7 kPa) at 30° C., a gave a yellow oil which was taken up in 1000 cm³ of ethyl acetate and 1000 cm³ of distilled water. The mixture obtained was brought to pH 2 by addition, with stirring, of concentrated hydrochloric acid and was then transferred into a separating funnel. The aqueous phase was separated out after settling had taken place and the organic phase was extracted with 200 cm³ of aqueous 0.1N hydrochloric acid solution. The aqueous phases were combined, washed again with 500 cm³ of ethyl acetate, placed in a round-bottomed flask with stirring and then basified to pH 7 by addition of sodium bicarbonate powder. The pH was then adjusted to pH 8 by addition of concentrated sodium hydroxide and the aqueous phase was extracted with two 600 cm³ portions of dichloromethane. The organic phase was separated out after settling had taken place, washed with 200 cm³ of distilled water, dried over magnesium sulfate, filtered and concentrated to dryness to give a solid which was stirred in 500 cm³ of diethyl ether and then filtered to give 57.8 g of a pale yellow powder. 30 g of this solid were purified by flash chromatography (eluent: 98/2 dichloromethane/methanol by volume), to give 8.2 g of a solid which was stirred for 1 hour in a mixture of 160 cm³ of ethyl acetate and diethyl ether (50/50 by volume) and 160 cm³ of 0.5N hydrochloric acid. The pH of this mixture was adjusted to pH 3-4 by addition of concentrated sodium hydroxide. The mixture obtained was separated by settling in a separating funnel. The aqueous phase was washed with a mixture of ethyl acetate and diethyl ether (50/50 by volume) and then basified to pH 8 by addition of sodium bicarbonate powder and extracted twice with ethyl acetate. The organic phases were combined, washed with water, dried over magnesium sulfate, filtered and then concentrated to dryness under reduced pressure (2.7 kPa) at 30° C., to give 7.2 g of a pale yellow solid which was stirred in 500 cm³ of diethyl ether for 18 hours, filtered, rinsed twice with diethyl ether and then dried at 20° C. 6.6 g of 5γ(S),5δ(R)[5γa,5δb]-1,4-hexahydrothiazepinopristinamycin IE were thus obtained in the form of a white powder which melted at 212° C.

¹H NMR spectrum, 400 MHz, CDCl₃

0.95 (m, 4H, CH₃ at 2γ and 5β); 1.3-1.45 (m, 5H, CH₃ at 1γ,3γ and 3β); 1.6-1.9 (m, 4H, 2×2β,365 and 5δ); 2.05 (m, 1H, 3β); 2.35-2.90 (m, 7H, 2×CH₂O of the 1,4-hexahydrothiazepine ring, 5β,5ε); 3 (s, 6H, N(Me)₂); 3.05-3.20 (m, 6H, 2×4β, NMe and 1H of the 1,4-hexahydrothiazepine ring); 3.30 (broad m, J at mid-height=11 Hz, 1H, 5γ); 3.45 (m, 2H, 3δ, and 1H of the CH₂N of the 1,4-hexahydrothiazepine ring); 3.5 (m, 1H, 3δ); 4.25 (broad d, J=15 Hz, 1H, 5ε); 4.6 (dd, J=8 and 6 Hz, 1H, 3α); 4.8 (m, 1H, 2α); 4.9 (m, 2H, 1α and 5α); 5.35 (t, 1H, 4α); 5.6 (d, J=8 Hz,1H, 6α); 5.9 (m, 1H, 1β); 6.62 (d, J=8 Hz, 2H, 4ε); 6.68 (d, J=9 Hz,1H, 2NH); 6.96 (d, J=8 Hz, 2H, 4δ); 7.2-7.4 (m, 7H, H₄, H₅ and aromatics at 6); 7.82 (dd, J=5 and 2 Hz, 1H, H₆); 8.52 (m, 2H, 1NH and 6NH); 11.7 (s, 1H, OH).

Crude 5δ-(2-aminoethyl)thiomethylpristinamycin IA was obtained in the following manner.

1.58 g of 2-aminoethanethiol was added, under a nitrogen atmosphere, to 12 g of 5δ-methylenepristinamycin IA dissolved in a mixture of 60 cm³ of dichloromethane and 20 cm³ of methanol. After 1.5 hours at 20° C., the reaction mixture was concentrated to dryness under reduced pressure (2.7 kPa), at 30° C. The residue obtained was stirred for 3 hours at 20° C. in 60 cm³ of distilled water. The suspension obtained was filtered through a sinter funnel. The solid obtained was washed with distilled water and then three times with diethyl ether. After drying in a desiccator at 45° C., 10.1 g of crude 5δ-(2-aminoethyl)thiomethyl-prisinamycin 1A were obtained in the form of a pale yellow powder, which was used without further purification.

### EXAMPLE 2

### 5γ(R),5δ(S)[5γa,5δb]-1,4-hexahydrothiazepinopristinamycin IE

9 g of crude 5δ-(2-aminoethylthio)methylpristinamycin IA was dissolved in 300 cm³ of acetonitrile at 50° C. After cooling, 30 cm³ of acetic acid and then 730 mg of sodium cyanoborohydride were added with stirring. After stirring for 52 hours, the solvent was evaporated off under reduced pressure (2.7 kPa at 30° C.). The thick oil obtained was taken up in 150 cm³ of ethyl acetate and 80 cm³ of distilled water. The mixture obtained was stirred at 20° C. and concentrated sodium hydroxide was then added until the pH was 7-8. After stirring for 15 minutes, the mixture was transferred into a separating funnel. The aqueous phase was separated out by settling and the organic phase was washed twice with 30 cm³ of distilled water containing sodium chloride. The organic phase was dried over magnesium sulfate, filtered and then concentrated to dryness (2.7 kPa at 30° C.) to give 9 g of a solid which was stirred in 180 cm³ of isopropyl ether for 2 hours. The solid obtained was filtered off, washed with diethyl ether and then dried to give 7.5 g of a pale yellow powder which was purified by flash chromatography (eluent: 95/5 dichloromethane/methanol by volume). 2.1 g of 5γ(S),5δ(R)[5γa,5δb]-1,4-hexahydrothiazepinopristinamycin IE, which is identical to the product described in Example 1, and 1.4 g of impure 5γ(R),5δ(S)[5γa,5δb]-1,4-hexahydrothiazepinopristin-amycin IE were thus obtained. The latter compound was taken up in 30 cm³ of diethyl ether, stirred overnight, filtered and then dried at 20° C., after which it was purified again by flash chromatography (eluent: 97/3 dichloromethane/methanol by volume) and gave 360 mg of 5γ(R),5δ(S)[5γa,5δb]-1,4-hexahydrothiazepino-pristinamycin IE in the form of a pale yellow powder which melted at 260° C.

¹H NMR spectrum, 400 MHz, CDCl₃

1 (t, 3H, CH₃ at 2γ); 1.14 (ddd, J=17, 12 and 5 Hz, 1H, 5β), 1.35 (m, 4H, CH₃ at 1γ and 362 ); 1.5 (m, 1H, 3γ); 1.65-1.75 (m, 2H, 2β); 2.05 (m, 1H, 3β); 2.34 (broad dd, J=17 and 4 Hz, 5β); 2.5 (m, 2H, CH₂S of the 1,4-hexahydrothiazepine ring and 5δ); 2.75 (m, 3H, 2H of the 1,4-hexahydrothiazepine ring and 5ε); 2.9-3.1 (m, 13H, N(CH₃)₂, NMe, 4β and 3 H of the 1,4-hexahydrothiazepine ring); 3.22 (m, 2H, 4β and CH₂N of the 1,4-hexahydrothiazepine ring); 3.4-3.60 (m, 3H, 3δ and 5γ); 4.6 (m, 2H, 3α and 5ε); 4.7 (m, 1H, 2α); 4.90 (dd, J=10 and 1.5 Hz, 1H, 1α); 5.10 (broad singlet, 1H, 5α); 5.52 (dd, J=10 and 8 Hz, 1H, 4α); 5.64 (d, J=8 Hz, 1H, 6α); 5.9 (m, 1H, 1β); 6.53 (d, J=8 Hz, 2H, 4δ); 6.72 (d, J=10 Hz, 1H, 2NH): 6.90 (d, J=8 Hz, 2H, 4ε); 7.08 (dd, J=8 and 5 Hz, 1H, H₅); 7.20 (dd, J=8 and 1.5 Hz, 1H, H₄); 7.35 (m, 5H aromatics at 6); 7.78 (dd, J=5 and 1.5 Hz, 1H, H₆); 8.52 (d, J=10 Hz, 1H, 1 NH); 8.78 (d, J=8 Hz, 1H, 6NH); 11.72 (s, 1H, OH).

### EXAMPLE 3

### 4ε-Chloro-5γ(S),5δ(R)[5γa,5δb]-1,4-hexahydrothiazepino-pristinamycin 1E

Working as in Example 1, but starting with 12.7 g of crude 4ε-chloro-5δ-(2-aminoethylthio)methylpristinamycin IA, molecular sieves, 300 cm³ of acetonitrile, and 30 cm³ of acetic acid, then stirring for 2 hours at 20° C., 955 mg of sodium cyanoborohydride were added. The stirring was continued for 18 hours. The reaction mixture was filtered, washed with acetonitrile, and concentrated to dryness under reduced pressure (2.7 kPa) at 30° C. An orange-colored solid was obtained. This product was taken up in 300 cm³ of ethyl acetate and 300 cm³ of distilled water and then treated as described in Example 1 and gave 6.6 g of a pale yellow powder which was purified by flash chromatography (eluent: 98/2 dichloromethane/methanol by volume). 1.3 g of impure 4ε-chloro-5γ(S),5δ(R)[5γa,5δb]-1,4-hexahydrothiazepinopristinamycin IE and 910 mg of impure 4ε-chloro-5γ(R),5δ(S)[5γa,5δb]-1,4-hexahydrothiazepino-pristinamycin IE were obtained.

1.3 g of impure 4ε-chloro-5γ(S),5δ(R)[5γa,5δb]-1,4-hexahydrothiazepinopristinamycin IE were dissolved in 30 cm³ of a dichloromethane/methanol mixture (85/15 by volume) and 650 mg of silica was then added. The mixture obtained was stirred for 5 hours at 20° C. and then filtered. The silica was rinsed with the same eluent and the filtrate was concentrated to dryness under reduced pressure (2.7 kPa) at 30° C. The solid obtained was stirred in diethyl ether, filtered and then dried and gave 1.26 g of a white powder which was purified by flash chromatography (eluent: 98/2 dichloromethane/methanol by volume). 810 mg of a solid was thus obtained and was stirred in 20 cm³ of diethyl ether, filtered and then dried under reduced pressure (30 Pa) at 20° C., to give 610 mg of 4ε-chloro-5γ(S),δ(R)[5γa,5δb]-1,4-hexahydrothiazepinopristinamycin IE in the form of a white solid which melted at 224° C.

¹H NMR spectrum, 400 MHz, CDCl₃

0.92 (t, 3H, CH₃ at 2γ); 1.24 (m, 2H, 3β and 5β); 1.32 (d, 3H, CH₃ at 1γ); 1.40 (m, 1H, 3γy); 1.55-1.70 (m, 5H, 2×2β,3γ and 5δ); 1.96 (m, 1H, 3β); 2.36 (dd, J=10 and 12 Hz, 1H, CH₂S of the 1,4-hexahydrothiazepine ring), 2.5-2.9 (m, 13H, 5β,5ε, N(Me)₂ and 4H of the 1,4-hexahydrothiazepine ring); 2.95-3.20 (m, 5H, 4β and NMe); 3.35 (m, 3H, 3δ, 5γ and 1H of CH₂N of the 1,4-hexahydrothiazepine ring); 3.5 (m, 1H, 3δ); 4.20 (broad d, J=15 Hz, 1H, 5ε); 4.52 (dd, J=8 and 6 Hz, 1H, 3α); 4.78 (m, 1H, 2α); 4.86 (d, J=10 Hz, 1H, 1α); 5 (broad d, J=6 Hz, 1H, 5α); 5.40 (dd, J=8 and 10 Hz, 1H, 4α); 5.56 (d, J=8 Hz, 1H, 6α); 5.9 (m, 1H, 1β); 6.64 (d, J=10 Hz, 1H, 2NH); 6.80 (d, J=8 Hz, 1H, 4ε); 6.84 (broad d, J=8 Hz, 1H, 4δ); 7.05 (broad s, 1H, 4δ); 7.12 (dd, J=8 and 5 Hz, 1H, H5); 7.20 (d, J=8 Hz, 1 H, H₄); 7.30-7.40 (m, 5H, aromatics at 6); 7.68 (broad d, J=5 Hz, 1H, H₆); 8.35 (d, J=10 Hz, 1H, 1NH); 8.50 (d, J=8 Hz, 1H, 6NH); 11.7 (s, 1H, OH).

4ε-chloro-5δ-(2-aminoethyl)thiomethylpristinamycin IA was prepared in the following manner:

Crude 4ε-chloro-5δ-(2-aminoethyl)thiomethylpristinamycin IA was obtained as described in Example 2, starting with 11.7 g of 4ε-chloro-5δ-methylenepristinamycin IA and 1.48 g of 2-aminoethanethiol in a mixture of 60 cm³ of dichloromethane and 20 cm³ of methanol, at −20° C. for 6 hours and then at 20° C. for 18 hours. After treatment as in Example 2, a solid was obtained, which was stirred in 200 cm³ of diethyl ether, filtered and dried under reduced pressure (30 Pa) at 20° C., to give 12.7 g of crude 4ε-chloro-5δ-(2-aminoethyl)thiomethylpristinamycin IA, in the form of a pink powder which was used without further purification.

4ε-chloro-5δ-methylenepristinamycin IA was obtained in the following manner.

1.9 g of N-chlorosuccinimide was added, under an argon atmosphere, to 11.4 g of 5δ-methylenepristinamycin IA dissolved in 120 cm³ of acetonitrile. The mixture was stirred at reflux for 2 hours, followed by addition of a further 346 mg of N-chlorosuccinimide. After refluxing for a further 1.5 hours and stirring for 18 hours at 20° C., the reaction mixture was concentrated to dryness under reduced pressure (2.7 kPa), at 30° C. The solid obtained was stirred for 4 hours in 250 cm³ of diethyl ether, filtered, rinsed and then dried in a fume cupboard at 20° C. and gave 11.7 g of 4ε-chloro-5δ-methylenepristinamycin IA in the form of a pink powder used without further purification.

### EXAMPLE 4

### 5γ(S),5δ(R)-4-methyl-[5γa,5δb]-1,4-hexahydrothiazepino-pristinamycin IE

1.5 g of 5γ(S),5δ(R)[5γa,5δb]-1,4-hexahydrothiazepinopristinamycin IE dissolved in 45 cm³ of acetonitrile was placed in a round-bottomed flask, under an argon atmosphere, followed by successive addition of 121 mg of sodium cyanoborohydride, 286 mg of paraformaldehyde and 4.5 cm³ of acetic acid. After stirring for 18 hours at 20° C., the mixture was filtered and then concentrated to dryness (2.7 kPa) at 30° C. The solid obtained was taken up with stirring in 60 cm³ of ethyl acetate and 20 cm³ of distilled water. The mixture was acidified to pH 2 by addition of 15 cm³ of 1 N hydrochloric acid, stirred for 2.5 hours and then transferred into a separating funnel. The aqueous phase was extracted with 15 cm³ of ethyl acetate. The aqueous phases were combined and brought to pH 7 by slow addition, with stirring, of solid sodium bicarbonate. The pH was adjusted to pH 8 by addition of 1N sodium hydroxide and the aqueous phase was extracted with two 50 cm³ portions of ethyl acetate. The organic phases were combined, washed with 10 cm³ of distilled water, dried over magnesium sulfate, filtered and concentrated to dryness under reduced pressure (2.7 kPa) at 30° C. 1.2 g of a pale yellow powder was thus obtained, and was purified by flash chromatography (eluent: 97/3 dichloromethane/methanol by volume) to give a solid which was taken up in diethyl ether, filtered and dried under reduced pressure (30 Pa) at 20° C. 620 mg of 5γ(S),5δ(R)-4-methyl-[5γa,5δb]-1,4-hexahydrothiazepinopristinamycin IE was thus obtained in the form of a pale yellow solid which melted at 202° C.

Mass spectrum: DCI (NH₃) m/z=954, MH⁺

### EXAMPLE 5

### 5γ(R),5δ(S)4-Methyl-[5γa,5δb]-1,4-hexahydrothiazepino-pristinamycin IE

Working as in Example 4, but starting with 410 mg of impure 5γ(R),5δ(S)[5γa,5δb]-1,4-hexahydrothiazepinopristinamycin IE in 13 cm³ of acetonitrile, 33 mg of sodium cyanoborohydride, 78 mg of paraformaldehyde and 1.3 cm³ of acetic acid, and after stirring for 18 hours at 20° C., 380 mg of a white powder was obtained, which was purified by flash chromatography (eluent: 97/3 dichloromethane/methanol by volume) and gave 230 mg of 5γ(R),5δ(S)-4-methyl-[5γa,5δb]-1,4-hexahydrothiazepinopristinamycin IE in the form of a pale yellow powder which melted at 200° C.

¹H NMR spectrum, 400 MHz, CDCl₃

0.98 (t, 3H, CH₃ at 2γ); 1.8-1.9 (m, 5H, CH₃ at 1γ,3β, and 5β); 1.52 (m, 1H, 3γ); 1.65-1.85 (m, 3H, 2β and 3γ); 2.04 (m, 1H, 3β); 2.45-2.60 (m, 6H, NCH₃ and 1H of the CH₂S of the 1,4-hexahydrothiazepine ring, 5β and 5δ); 2.7 (dd, J=17 and 5 Hz, 1H, 5ε); 2.75-2.95 (m, 4H, 4H of the 1,4-hexahydrothiazepine ring); 2.96 (s, 6H, N(CH₃)₂); 3.04-3.28 (m, 7H, 1H of NCH₂ of the 1,4-hexahydrothiazepine ring, 4β,5γ and NMe); 3.42-3.58 (m, 2H, 3δ); 4.58 (dd, J=8 and 6 Hz,1H, 3α); 4.66 (broad doublet, J=17 Hz, 1H, 5ε); 4.90 (dd, J=10 and 1.5 Hz, 1H, 1α); 5.18 (broad singlet, 1H, 5α); 5.60 (dd, J=10 and 8 Hz, 1H, 4α); 5.68 (d, J=10 Hz, 1H, 6α); 5.94 (m, 1H, 1β); 6.54 (d, J=8 Hz, 2H, 4ε); 6.76 (d, J=8 Hz, 1H, 2NH); 6.92 (d, J=8 Hz, 2H, 4δ); 7.08 (dd, J=8 and 5 Hz, 1H, H₅); 7.20 (dd, J=8 and 1.5 Hz, 1H, H₄); 7.3-7.4 (m, 5H, aromatics at 6); 7.78 (dd, J=5 and 1.5 Hz, 1H, H₆); 8.56 (d, J=10 Hz, 1H, 1NH); 8.80 (d, J=8 Hz, 1H, 6NH); 11.76 (s, 1H, OH).

### EXAMPLE 6

### 5γ(S),5δ(R)-4-Ethyl-[5γa,5δb]-1,4-hexahydrothiazepino-pristinamycin IE

Working as in Example 4, but starting with 1.3 g of impure 5γ(S),5δ(R)[5γa,5δb]-1,4-hexahydrothiazepinopristinamycin IE in 39 cm³ of acetonitrile, 105 mg of sodium cyanoborohydride, 310 mg of acetaldehyde and 3.9 cm³ of acetic acid, and after stirring for 2.5 hours at 20° C., 1.1 g of a pale yellow powder was obtained and was purified by flash chromatography (eluent: 97/3 dichloromethane/methanol by volume) to give a solid which was stirred in 16 cm³ of diethyl ether, filtered and dried under reduced pressure (30 Pa) at 20° C. 690 mg of 5γ(S),5δ(R)-4-ethyl-[5γa,5δb]-1,4-hexahydrothiazepinopristinamycin IE was thus obtained in the form of a pale yellow powder which melted at 202° C.

Mass spectrum: FAB (NBA matrix) m/z=968, MH⁺

### EXAMPLE 7

### 5γ(S),5δ(R)-2,2-Dimethyl-[5γa,5δb]-1,4-hexahydrothiazepino-pristinamycin IE

### 5γ(R),5δ(S)-2,2-Dimethyl-[5γa,5δb]-1,4-hexahydrothiazepino-pristinamycin IE

Working as in Example 1, but starting with 31 g of crude 5δ-[(1-methyl)aminopropyl]thiomethylpristinamycin IA, 780 cm³ of acetonitrile, 78 cm³ of acetic acid and 2.43 g of sodium cyanoborohydride, and after stirring for 18 hours at 20° C., followed by treatment, 25.65 g of a solid was obtained and was purified by flash chromatography (eluent: 98/2 dichloromethane/methanol by volume) and gave a solid which was dried under reduced pressure (30 Pa) at 20° C. 8.3 g of 5γ(S),5δ(R)-2,2--dimethyl-[5γa,5δb]-1,4-hexahydrothiazepinopristinamycin IE was thus obtained in the form of a pale yellow solid which melted at 210° C.

¹H NMR spectrum, 600 MHz, CDCl₃

0.90 (ddd, J=17.6 and 5 Hz, 1H, 5β); 0.94 (m, 3H, CH₃ at 2γ); 1.12 (s, 3H, CH₃); 1.28-1.45 (m, 8H, CH₃ at 1γ,3β, and 3γ); 1.62-1.82 (m, 4H, 2β,3γ and 5δ); 2 (m, 1H, 3β); 2.38 (broad s, 1H, NH), 2.42 (d, J=17 Hz, 5β); 2.46 (dd, 1H, 1H of SCH₂ of the 1,4-hexahydrothiazepine ring); 2.55 (m, 3H, 2H of the 1,4-hexahydrothiazepine ring and 5ε); 2.8 (d, 1H,1H of NCH₂ of the 1,4-hexahydrothiazepine ring); 2.96 (s, 6H, N(Me)₂); 3-3.15 (m, 7H, NMe, 5γ and 4β); 3.36 (m, 1H, 3δ); 3.5 (m, 1H, 3δ); 4.2 (broad d, J=17 Hz, 1H, 5ε); 4.58 (dd, J=8 and 6 Hz, 1H, 3α); 4.78 (m, 1H, 2α); 4.85 (m, 2H, 1α and 5α); 5.28 (t, 1H, 4α); 5.54 (d, J=8 Hz, 1H, 6α); 5.86 (m, 1H, 1β); 6.60 (d, J=8 Hz, 2H, 4ε) 6.64 (d, J=8 Hz, 1H, 2NH); 6.94 (d, J=8 Hz, 2H, 4δ); 7.22 (dd, J=8 and 5 Hz, 1H, H₅); 7.27-7.37 (m, 6H, H₄ and aromatics at 6); 7.8 (dd, J=5 and 1.5 Hz, 1H, H₆); 8.48 (m, 2NH, 6NH and 1 NH); 11.68 (s,1H, OH).

In the same chromatography, 1.85 g of 5γ(R),5δ(S)-2,2-dimethyl-[5γa,5δb]-1,4-hexahydrothiazepinopristinamycin IE was isolated in the form of a pale yellow solid which melted at 202° C.

¹H NMR spectrum, 600 MHz, CDCl₃

0.86 (ddd, J=17, 12 and 5 Hz, 1H, 5β); 0.95 (t, 3H, CH₃ at 2γ); 1.15 (s, 3H, CH₃); 1.35 (m, 4H, CH₃ at 1γ and 3γ); 1.45 (m, 4H, CH₃ and 3β); 1.6-1.8 (m, 3H, 2β and 3γ); 2.14 (broad dd, J=17and 4 Hz, 1H, 5β); 2.2 (d, J=15 Hz, 1H, 1H of NCH₂ of the 1,4-hexahydrothiazepine ring); 2.45 (broad s, width at mid-height 10 Hz, 1H, 5δ); 2.7 (m, 2H, 1H of CH₂S of the 1,4-hexahydrothiazepine ring and 5ε); 2.9-3 (m, 8H, N(Me)₂, 2H of the 1,4-hexahydrothiazepine ring), 3.05 (dd, 1H, 4β); 3.08 (s, 3H, NMe); 3.15 (dd, 1H, 4β); 3.4 (m, 1H, 3δ); 3.52 (m, 2H, 3δ and 5γ); 4.54 (dd, J=8 and 6 Hz, 1H, 3α); 4.62 (broad d, J=15 Hz, 1H, 5ε); 4.88 (dd, J=10 and 1.5 Hz, 1H, 1α); 4.98 (broad s, 1H, 5α); 5.38 (dd, J=10 and 8 Hz, 1H, 4α); 5.62 (d, J=8 Hz, 1H, 6α) 5.88 (m, 1H, 1β); 6.56 (d, J=8 Hz, 2H, 4ε); 6.7 (d, J=8Hz, 1H, 2NH); 6.84 (d, J=8 Hz, 2H, 4δ); 7.15 (dd, J=8and 5 Hz, 1H, H₅); 7.24 (dd, J=8 and 1.5 Hz, 1H, H₄); 7.28-7.40 (m, 5H, aromatics at 6); 7.8 (dd, J=5 and 1.5 Hz, 1H, H₆); 8.56 (d, J=10 Hz, 1H, 1NH); 8.76 (d, J=8 Hz, 1H, 6NH); 11.72 (s, 1H, OH).

Crude 5δ-[(1-methyl)aminopropyl]thiomethylpristinamycin IA was obtained as described below by analogy with Example 2.

5.49 g of 1-amino-2-methyl-2-propanethiol hydrochloride and 5.1 cm³ of triethylamine were added at −30° C., under a nitrogen atmosphere, to 30 g of 5δ-methylenepristinamycin IA dissolved in a mixture of 150 cm³ of dichloromethane and 45 cm³ of methanol. After stirring for 7.5 hours at a temperature of from −20 to −15° C., the reaction mixture was concentrated to dryness under reduced pressure (2.7 kPa) at 30° C. The residue obtained was taken up in 500 cm³ of distilled water and 500 cm³ of dichloromethane. The aqueous phase was separated out after settling of the phases had taken place and was then extracted with 300 cm³ of dichloromethane. The organic phases were combined, washed successively with 500 cm³ of distilled water and 200 cm³ of distilled water saturated with sodium chloride, and then dried over magnesium sulfate, filtered and concentrated to dryness under reduced pressure (2.7 kPa) at 30° C., and gave a solid which was stirred in 300 cm³ of diethyl ether. After filtration, the solid obtained was dried (30 Pa) at 30° C. to give 31.3 g of crude 5δ-[(1-methyl)aminopropyl]thiomethylpristinamycin IA in the form of a cream-colored powder which was used without further purification.

### EXAMPLE 8

5γ(S), 5δ(R)-2,2,4-Trimethyl-[5γa, 5δb]-1,4-hexahydrothiazepino-pristinamycin IE

Working as in Example 4, but starting with 1.5 g of impure 5γ(S), 5δ(R)-2,2-dimethyl-[5γa, 5δb]-1,4-hexahydrothiazepinopristinamycin IE in 3 cm³ of acetonitrile, 118 mg of sodium cyanoborohydride, 278 mg of paraformaldehyde and 0.3 cm³ of acetic acid, and after stirring for 17.5 hours at 20° C., 1.13 g of a white powder was obtained and was purified by flash chromatography (eluent: 98/2 dichloromethane/methanol by volume) to give 459 mg of 5γ(S), 5δ(R)-2,2,4-trimethyl-[5γa, 5δb]-1,4-hexahydrothiazepinopristinamycin IE in the form of a white powder which melted at 220° C.

Mass spectrum: DCI (NH₃) m/z=981, MH⁺

### EXAMPLE 9

### 5γ(S), 5δ(R)-2,2-Dimethyl-4-(4-hydroxybutyryl)[5γa, 5δb]-1,4-hexahydrothiazepinopristinamycin IE

2.85 g of 5γ(S), 5δ(R)-2,2-dimethyl-4-(4-bromobutyryl)[5γa,5δb]-1,4-hexahydrothiazepinopristinamycin IE dissolved in 100 cm³ of dimethylformamide and 0.44 cm³ of morpholine was placed in a round-bottomed flask maintained under a nitrogen atmosphere, at 24° C. After stirring for 4.5 hours, the mixture was poured onto 1000 cm³ of distilled water and 500 cm³ of dichloromethane. The organic phase was separated out after settling of the phases has taken place, extracted with two portions of 500 cm³ of distilled water and then with 500 cm³ of saturated sodium chloride solution, dried over magnesium sulfate, filtered and concentrated to dryness under reduced pressure (2.7 kPa) at 30° C., and gave 2.6 g of an oil. This residue was purified by flash chromatography (eluent: 97/3 dichloromethane/methanol by volume) and gave a solid which was stirred in diethyl ether, filtered and dried under reduced pressure (30 Pa) at 20° C. 140 mg of 5γ(S), 5δ(R)-2,2-dimethyl-4-(4-hydroxybutyryl)[5γa, 5δb]-1,4-hexahydrothiazepinopristinamycin IE was thus obtained in the form of a solid which melted at 194° C.

Mass spectrum: FAB (NBA matrix) m/z=1054, MH⁺

5γ(S), 5δ(R)-2,2-dimethyl-4-(4-bromobutyryl)[5γa, 5δb]-1,4-hexahydrothiazepinopristinamycin IE was prepared in the following manner:

2 g of 5γ(S), 5δ(R)-2,2-dimethyl-[5γa, 5δb]-1,4-hexahydrothiazepinopristinamycin IE, 80 cm³ of dichloromethane over amylene and 0.44 cm³ of triethylamine were placed in a round-bottomed flask at −10° C., followed by dropwise addition, over 1 hour 10 minutes, of 0.378 cm³ of 4-bromobutyryl chloride dissolved in 20 cm³ of dichloromethane over amylene. After stirring for 22 hours at 20° C., 0.146 cm³ of triethylamine and 0.126 cm³ of 4-bromobutyryl chloride were added, at 0° C. The reaction mixture was stirred for a further 18 hours at 20° C. and was then poured into 40 cm³ of distilled water. The mixture obtained was separated by settling of the phases and the organic phase was washed successively with 20 cm³ of distilled water and 20 cm³ of water saturated with sodium chloride. The resulting organic phase was dried over magnesium sulfate, filtered and concentrated to dryness under reduced pressure (2.7 kPa) at 30° C., and gave 2.89 g of 5γ(S), 5δ(R)-2,2-dimethyl-4-(4-bromobutyryl)[5γa, 5δb]-1,4-hexahydrothiazepino-pristinamycin IE in the form of an off-white solid which was used in crude form.

The present invention also relates to pharmaceutical compositions comprising at least one streptogramin derivative according to the invention, where appropriate in the form of a salt, alone or in combination with at least one compatible and pharmaceutically acceptable diluent or adjuvant. The invention also relates to the above pharmaceutical compositions further comprising at least one group A streptogramin derivative or, where appropriate, at least one of the salts thereof, combined with the at least one streptogramin derivative of formula (I) or salt thereof.

The compositions according to the invention can be used, for example, orally, parenterally, topically, rectally or as aerosols.

As used in the context of the present invention, the terms active product, active principle, and active ingredient are understood to mean at least one streptogramin derivative chosen from group B streptogramin derivatives, stereoisomers of group B streptogramin derivatives, salts thereof, alone or in combination with at least one group A streptogramin derivative.

Solid compositions for oral administration which can be used include, for example, tablets, pills, gel capsules, powders or granules. In these compositions, the active product, is mixed with at least one inert diluent or adjuvant, such as sucrose, lactose or starch. These compositions can comprise substances other than diluents, for example, a lubricant such as magnesium stearate or a coating intended for controlled release.

Liquid compositions for oral administration which can be used include, for example, pharmaceutically acceptable solutions, suspensions, emulsions, syrups and elixirs comprising inert diluents such as water or liquid paraffin. These compositions can also comprise substances other than diluents, for example wetting, sweetening or flavoring products.

Compositions for parenteral administration may comprise, for example, sterile solutions or emulsions. Solvents or vehicles which may be used include propylene glycol, polyethylene glycol, plant oils, such as olive oil, and injectable organic esters, for example ethyl oleate. Compositions can further comprise at least one adjuvant, such as wetting agents, isotonic agents, emulsifiers, dispersants and stabilizers.

Sterilization can be carried out in several ways, for example using a bacteriological filter, by irradiation or by heating. The compositions according to the invention can also be prepared in the form of sterile solid compositions which may be dissolved at the time of use in sterile water or in any other injectable sterile medium.

Compositions for topical administration may comprise, for example, creams, ointments, lotions or aerosols.

Compositions for rectal administration can be in the form of suppositories or rectal capsules, which comprise, besides the active principle, excipients such as cocoa butter, semisynthetic glycerides or polyethylene glycols.

The compositions according to the invention may also be aerosols. For use in the form of liquid aerosols, the compositions may be stable sterile solutions or solid compositions that are dissolved at the time of use in apyrogenic sterile water, in saline or in any other pharmaceutically acceptable vehicle. For use in the form of dry aerosols intended to be inhaled directly, the active principle can be finely divided and combined with a water-soluble solid vehicle or diluent with a particle size ranging, for example, from 30 μm to 80 μm, for example dextran, mannitol or lactose.

In human therapy, the novel streptogramin derivatives according to the invention can be useful, for example, in the treatment of infections of bacterial origin. The doses depend on the desired effect and on the duration of the treatment. The doctor will determine the dosage s/he considers to be the most suitable as a function of the treatment, depending on the age, weight, degree of infection and other factors specific to the individual to be treated. Generally, the doses range, for example, from 1 g to 3 g of active product taken 2 or 3 times a day, orally for an adult.

The example which follows illustrates a composition according to the invention.

### EXAMPLE

Tablets comprising a 250 mg dose of active product and having the composition below were prepared according to known, art-recognized techniques:

| | |
|---|---|
| - 5γ(S),5δ(R)[5γa,5δb]-1,4- | 75 mg |
| hexahydrothiazepinopristinamycin IE | |
| - pristinamycin II_{B} | 175 mg |
| - excipient: starch, hydrated silica, dextrin, gelatin, | qs 500 mg |
| magnesium stearate: | |

What is claimed is:

1. A group B streptogramin derivative of formula (I) or a salt thereof: 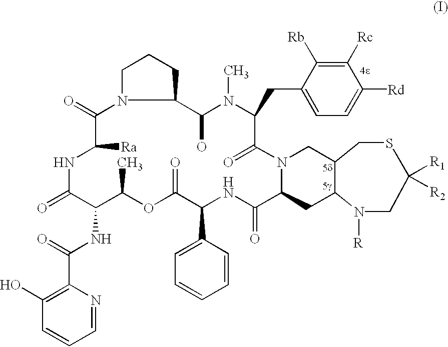
wherein:
R is chosen from a hydrogen atom, a methyl group, alkyl groups of formula R′—CH₂—, wherein R′ is chosen from straight and branched alkyl groups, and acyl groups unsubstituted or substituted with a hydroxyl group,
R₁ and R₂, which are identical or different, are each chosen from a hydrogen atom and alkyl groups,
Ra is chosen from a methyl group and an ethyl group, and
Rb, Rc and Rd are defined as follows:
1) Rb and Rc are each a hydrogen atom, and Rd is chosen from a hydrogen atom, a methylamino group, and a dimethylamino group, or
2) Rb is a hydrogen atom, Rc is chosen from a hydrogen atom, a chlorine atom, a bromine atom, and C₃-C₅ alkenyl groups, and Rd is chosen from —N(CH₃)—R′″ groups, wherein R′″ is chosen from:
(a) alkyl groups,
(b) C₂-C₄ hydroxyalkyl groups,
(c) unsubstituted C₂-C₈ alkenyl groups,
(d) C₂-C₈ alkenyl groups substituted with (i) an unsubstituted or substituted phenyl group, (ii) an unsubstituted or substituted cycloalkyl(C₃-C₆)methyl group, (iii) an unsubstituted benzyl group, (iv) a benzyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups, or (v) heterocyclylmethyl groups and heterocyclylethyl groups, wherein the heterocyclyl portions of said heterocyclylmethyl groups and said heterocyclylethyl groups are chosen from saturated and unsaturated 5-or 6-membered heterocyclyl groups comprising from 1 or 2 heteroatoms chosen from a sulphur atom, an oxygen atom, and a nitrogen atom, and wherein said heterocyclyl groups are unsubstituted or substituted with a group chosen from alkyl groups, C₂-C₈ alkenyl groups, C₃-C₆ cycloalkyl groups, saturated and unsaturated 4- to 6-membered heterocyclyl groups, an unsubstituted phenyl group, a benzyl group, or a phenyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups,
(e) a cyanomethyl group,
(f) a carboxymethyl group, and
(g) —CORe groups and —CH₂CORe groups, wherein Re is chosen from (I)-OR′e groups, wherein R′e is chosen from C₁-C₆ alkyl groups, C₂-C₆ alkenyl groups, a benzyl group, and heterocyclylmethyl groups, wherein said heterocyclyl portion is chosen from 5- or 6- membered heterocyclyl groups comprising from 1 or 2 heteroatoms chosen from a sulphur atom, an oxygen atom, and a nitrogen atom, (ii) alkylamino groups, (iii) alkylmethylamino groups, (iv) heterocyclylamino groups and heterocyclylmethylamino groups, wherein said heterocyclyl portion of said heterocyclylamino groups and said heterocyclylmethylamino groups is chosen from 5- or 6-membered saturated heterocyclyl groups comprising from 1 or 2 heteroatoms chosen from a sulphur atom, an oxygen atom, and a nitrogen atom, and wherein said heterocyclyl groups are unsubstituted or substituted with a group chosen from alkyl groups, a benzyl group, and alkyloxycarbonyl groups, or
3) Rb is a hydrogen atom, Rd is chosen from an —NHCH₃ group and an —N(CH₃)₂ group, and Rc is chosen from a chlorine atom and a bromine atom, and when Rd is an —N(CH₃)₂ group, Rc is chosen from C₃-C₅ alkenyl groups, or
4) Rb and Rd are each a hydrogen atom, and Rc is chosen from halogen atoms, alkylamino groups, dialkylamino groups, alkyloxy groups, a trifluoromethoxy group, thioalkyl groups, C₁-C₆ alkyl groups, and trihalomethyl groups, or
5) Rb and Rc are each a hydrogen atom, and Rd is chosen from halogen atoms, an ethylamino group, a diethylamino group, a methylethylamino group, alkyloxy groups, a trifluoromethoxy group, alkylthio groups, alkylsulfinyl groups, alkylsulfonyl groups, C₁-C₆ alkyl groups, a phenyl group, and trihalomethyl groups, or
6) Rb is a hydrogen atom, Rc is chosen from halogen atoms, alkylamino groups, dialkylamino groups, alkyloxy groups, a trifluoromethoxy group, thioalkyl groups, and C₁-C₃ alkyl groups, and Rd is chosen from halogen atoms, an amino group, alkylamino groups, dialkylamino groups, alkyloxy groups, a trifluoromethoxy group, thioalkyl groups, C₁-C₆ alkyl groups, and trihalomethyl groups, or
7) Rc is a hydrogen atom, and Rb and Rd are each a methyl group, unless stated otherwise, said alkyl groups are chosen from straight and branched, unsubstituted or substituted C₁-C₄ alkyl groups, and
unless stated otherwise, said acyl groups are chosen from straight and branched, unsubstituted or substituted C₁-C₄ acyl groups,
and wherein said group B streptogramin derivative of formula (I) is chosen from 5γ(R), 5δ(S) group B streptogramin derivatives, salts thereof, 5γ(S), 5δ(R) group B streptogramin derivatives, salts thereof, and mixtures of any of the foregoing.

2. A group B streptogramin derivative according to claim 1, wherein:
R is chosen from a hydrogen atom, a methyl group, alkyl groups of formula R′—CH₂—, wherein R′ is chosen from straight and branched alkyl groups, and acyl groups unsubstituted or substituted with a hydroxyl group,
R₁ and R₂, which are identical or different, are each chosen from a hydrogen atom and alkyl groups,
Ra is an ethyl group, and
Rb, Rc, and Rd are defined as follows:
1) Rb and Rc are each a hydrogen atom, and
Rd is chosen from a methylamino group and a dimethylamino group, or
2) Rb is a hydrogen atom,
Rd is chosen from an —NHCH₃ group and an —N(CH₃)₂ group, and
Rc is a chlorine atom.

3. A group B streptogramin derivative according to claim 1, wherein said streptogramin is 5γ(S), 5δ(R)[5γa, 5δb]-1,4-hexahydrothiazepinopristinamycin IE.

4. A group B streptogramin derivative according to claim 1, wherein said streptogramin is 4ε-chloro-5γ(S),5δ(R)[5γa,5δb]-1,4-hexahydrothiazepinopristinamycin IE.

5. A group B streptogramin derivative according to claim 1, wherein said streptogramin is 5γ(R), 5δ(S)-2,2-dimethyl-[5γa, 5δb]-1,4-hexahydrothiazepinopristinamycin IE.

6. A group B streptogramin derivative according to claim 1, wherein said streptogramin is 5γ(S), 5δ(R)-2,2-dimethyl-4-(4-hydroxybutyryl)[5γa, 5δb]-1,4-hexahydrothiazepino-pristinamycin IE.

7. A process for preparing at least one group B streptogramin derivative of formula (I) according to claim 1, said process comprising:
A) reacting an amino mercaptan of formula (II): 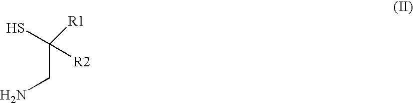
wherein
R₁ and R₂, which are identical or different, are each chosen from a hydrogen atom and alkyl groups,
with a streptogramin derivative of formula (III): 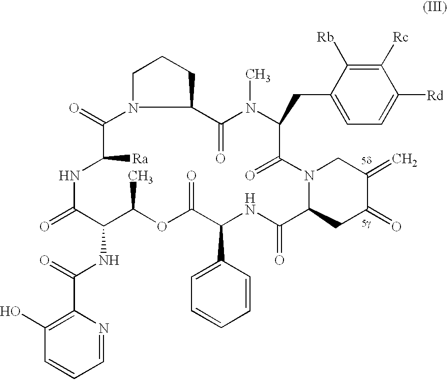
wherein
Ra is chosen from a methyl group and an ethyl group, and
Rb, Rc and Rd are defined as follows:
1) Rb and Rc are each a hydrogen atom, and Rd is chosen from a hydrogen atom, a methylamino group, and a dimethylamino group, or
2) Rb is a hydrogen atom, Rc is chosen from a hydrogen atom, a chlorine atom, a bromine atom, and C₃-C₅ alkenyl groups, and Rd is chosen from —N(CH₃)—R′″ groups, wherein R′″ is chosen from:
(a) alkyl groups,
(b) C₂-C₄ hydroxyalkyl groups,
(c) unsubstituted C₂-C₈ alkenyl groups,
(d) C₂-C₈ alkenyl groups substituted with (i) an unsubstituted or substituted phenyl group, (ii) an unsubstituted or substituted cycloalkyl(C₃-C₆)methyl group, (iii) an unsubstituted benzyl group, (iv) a benzyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups, or (v) heterocyclylmethyl groups and heterocyclylethyl groups, wherein said heterocyclyl portions of said heterocyclylmethyl groups and said heterocyclylethyl groups are chosen from saturated and unsaturated 5-or 6-membered heterocyclyl groups comprising from 1 or 2 heteroatoms chosen from a sulphur atom, an oxygen atom, and a nitrogen atom, and wherein said heterocyclyl groups are unsubstituted or substituted with a group chosen from alkyl groups, C₂-C₈ alkenyl groups, C₃-C₆ cycloalkyl groups, saturated and unsaturated 4- to 6-membered heterocyclyl groups, an unsubstituted phenyl group, a benzyl group, or a phenyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups,
(e) a cyanomethyl group,
(f) a carboxymethyl group, and
(g) a —CORe groups and —CH₂CORe groups, wherein Re is chosen from (i) —OR′e groups, wherein R′e is chosen from C₁-C₆ alkyl groups, C₂-C₆ alkenyl groups, a benzyl group, and heterocyclylmethyl groups, wherein said heterocyclyl portion is chosen from 5- or 6- membered heterocyclyl groups comprising from 1 or 2 heteroatoms chosen from a sulphur atom, an oxygen atom, and a nitrogen atom, (ii) alkylamino groups, (iii) alkylmethylamino groups, (iv) heterocyclylamino groups and heterocyclylmethylamino groups, wherein said heterocyclyl portion of said heterocyclylamino groups and said heterocyclylmethylamino groups is chosen from 5- or 6-membered saturated heterocyclyl groups comprising from 1 or 2 heteroatoms chosen from a sulphur atom, an oxygen atom, and a nitrogen atom, and wherein said heterocyclyl groups are unsubstituted or substituted with a group chosen from alkyl groups, a benzyl group, and alkyloxycarbonyl groups, or
3) Rb is a hydrogen atom, Rd is chosen from an —NHCH₃ group and an —N(CH₃)₂ group, and Rc is chosen from a chlorine atom and a bromine atom, and when Rd is an —N(CH₃)₂ group, Rc is chosen from C₃-C₅ alkenyl groups, or
4) Rb and Rd are each a hydrogen atom, and Rc is chosen from halogen atoms, alkylamino groups, dialkylamino groups, alkyloxy groups, a trifluoromethoxy group, thioalkyl groups, C₁-C₆ alkyl groups, and trihalomethyl groups, or
5) Rb and Rc are each a hydrogen atom, and Rd is chosen from halogen atoms, an ethylamino group, a diethylamino group, a methylethylamino group, alkyloxy groups, a trifluoromethoxy group, alkylthio groups, alkylsulfinyl groups, alkylsulfonyl groups, C₁-C₆ alkyl groups, a phenyl group, and trihalomethyl groups, or
6) Rb is a hydrogen atom, Rc is chosen from halogen atoms, alkylamino groups, dialkylamino groups, alkyloxy groups, a trifluoromethoxy group, thioalkyl groups, and C₁-C₃ alkyl groups, and Rd is chosen from halogen atoms, an amino group, alkylamino groups, dialkylamino groups, alkyloxy groups, a trifluoromethoxy group, thioalkyl groups, C₁-C₆ alkyl groups, and trihalomethyl groups, or
7) Rc is a hydrogen atom, and Rb and Rd are each a methyl group, to prepare at least one 5δ-aminoethylthiomethyl derivative,
B) reducing said at least one 5δ-aminoethylthiomethyl derivative prepared above in (A) to prepare at least one group B streptogramin derivative according to claim 1,
C) optionally separating said at least one group B streptogramin derivative,
D) optionally substituting, at the R position of formula (I), said at least one group B streptogramin derivative prepared in (B) or (C) above with an R group chosen from a hydrogen atom, a methyl group, alkyl groups of formula R′—CH₂—, wherein R′ is chosen from straight and branched alkyl groups, and acyl groups unsubstituted or substituted with a hydroxyl group, and
E) optionally converting at least one streptogramin derivative prepared in (B), (C), or (D) above to an acid addition salt.

8. A process according to claim 7, wherein said substitution with an R group comprises reacting, in a reductive medium, a corresponding streptogramin derivative for which R is a hydrogen atom with an aldehyde of formula (IV):
R—CHO (IV)
wherein R is chosen from a methyl group and alkyl groups of formula R′—CH₂—, wherein R′ is chosen from straight and branched alkyl groups, to obtain a group B streptogramin derivative of formula (I), wherein R is chosen from a methyl group and alkyl groups of formula R′—CH₂—, wherein R′ is chosen from straight and branched alkyl groups.

9. A process according to claim 7, wherein said substitution with an R group comprises acylating a corresponding streptogramin derivative for which R is a hydrogen atom by any known method that does not affect the rest of the molecule to obtain a group B streptogramin derivative of formula (I), wherein R is chosen from acyl groups unsubstituted or substituted with a hydroxyl group.

10. A pharmaceutical composition comprising at least one group B streptogramin derivative of formula (I) or salt thereof according to claim 1.

11. A pharmaceutical composition according to claim 10, further comprising at least one group A streptogramin derivative or salt thereof.

12. A pharmaceutical composition according to claim 10, further comprising at least one compatible agent chosen from pharmaceutically acceptable diluents and pharmaceutically acceptable adjuvants.

13. A pharmaceutical composition according to claim 11, further comprising at least one compatible agent chosen from pharmaceutically acceptable diluents and pharmaceutically acceptable adjuvants.

14. A pharmaceutical composition according to claim 11, wherein said at least one group A streptogramin derivative or salt thereof is chosen from (A) pristinamycin IIA, pristinamycin IIB, pristinamycin IIC, pristinamycin IID, pristinamycin IIE, pristinamycin IIF, and pristinamycin IIG, (B) semisynthetic group A streptogramin derivatives, (C) derivatives of formula (α) and salts thereof: 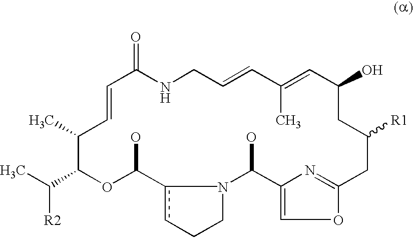
wherein:
R₁ is chosen from
(a) —NR′R″ groups, wherein R′ is chosen from a hydrogen atom and a methyl group, and R″ is chosen from a hydrogen atom, alkyl groups, cycloalkyl groups, an allyl group, a propargyl group, and a benzyl group,
(b) —OR′″ groups, wherein R′″ is chosen from a hydrogen atom, alkyl groups, cycloalkyl groups, an allyl group, a propargyl group, and a benzyl group, and
(c) —NR₃R₄ groups, wherein R₃ and R₄ are each a methyl group or form, together with the nitrogen atom to which they are attached, a saturated or unsaturated 4- or 5-membered heterocycle optionally comprising, in addition to said nitrogen atom, a hetero atom chosen from a nitrogen atom, an oxygen atom, and a sulfur atom,
R₂ is chosen from a hydrogen atom, a methyl group, and an ethyl group, and
the bond --- is a single bond or a double bond,
(D) semisynthetic derivatives of formula (β) and salts thereof: 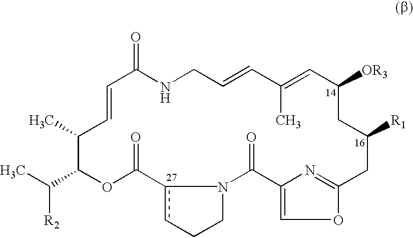
wherein:
R₁ is chosen from halogen atoms, an azido group, and a thiocyanato group,
R₂ is chosen from a hydrogen atom, a methyl group, and an ethyl group,
R₃ is chosen from a hydrogen atom and unsubstituted or substituted aliphatic ester residues, unsubstituted or substituted cycloaliphatic ester residues, unsubstituted or substituted aromatic ester residues, unsubstituted or substituted araliphatic ester residues, unsubstituted or substituted heterocyclic ester residues, and unsubstituted or substituted heterocyclylaliphatic ester residues, and
the bond --- is a single bond (27R stereochemistry) or a double bond.

15. A pharmaceutical composition according to claim 14, wherein said R₃ of said semisynthetic derivatives of formula (β) and salts thereof is an R′₃—CO— group, wherein R′₃ is chosen from:
(A) an unsubstituted or substituted phenyl group and unsubstituted or substituted phenylalkyl groups, wherein, when R′₃ is a substituted phenyl group or a substituted phenylalkyl group, the phenyl portion is substituted with at least one substituent chosen from
(1) alkyl groups, unsubstituted or substituted with an NR″R′″ group, wherein
(a) R″ and R′″, which are identical or different, are each chosen from a hydrogen atom and alkyl groups which can form, together with the nitrogen atom to which they are attached, a saturated or unsaturated 3- to 8-membered heterocyclyl group, optionally comprising, in addition to said nitrogen atom, another hetero atom chosen from an oxygen atom, a sulfur atom, and a nitrogen atom, wherein said heterocyclyl group is unsubstituted or substituted with at least one group chosen from saturated and unsaturated 3- to 8-membered alkyl groups, saturated and unsaturated 3- to 8-membered hydroxyalkyl groups, saturated and unsaturated 3- to 8-membered alkyloxyalkyl groups, saturated and unsaturated 3- to 8-membered alkyloxycarbonylalkyl groups, saturated and unsaturated 3- to 8-membered aryl groups, saturated and unsaturated 3- to 8-membered heterocyclyl groups, saturated and unsaturated 3- to 8-membered heterocyclylalkyl groups, and —CH₂—CO—NR″R′″ groups, wherein NR″R′″ is defined as above, or
(b) R″ and R′″, which are identical or different, are each chosen from (i) hydroxyalkyl groups, (ii) a phenyl group, and (iii) saturated and unsaturated 3- to 8-membered heterocyclylalkyl groups,
(2) alkyl groups unsubstituted or substituted with a —CO—NR″R′″ group, wherein NR″R′″ is defined as above,
(3) alkyl groups substituted with an NR″R′″ group as defined above, and
(4) acyl groups substituted with an NR″R′″ group as defined above,
(B) a substituted phenyl group and substituted phenylalkyl groups, wherein said phenyl group or phenyl portion of said phenylalkyl is substituted with at least one substituent chosen from (a) alkyl groups, unsubstituted or substituted with at least one group chosen from alkyloxy groups and alkylthio groups, wherein said alkyloxy groups or said alkylthio groups are unsubstituted or substituted with a carboxyl group or an NR″R′″ group as defined above, and (b) acyloxy groups which are unsubstituted or substituted with an NR″R′″ group as defined above,
(C) alkyl groups and cycloalkyl groups, wherein said alkyl groups and said cycloalkyl groups are unsubstituted or substituted with at least one group chosen from (a) a carboxyl group, (b) carboxyalkyldisulfanyl groups, (c) NR″R′″ groups, —CH₂—NR″R′″ groups, and —CO—NR″R′″ groups, wherein NR″R′″ is defined as above, (d) alkyloxycarbonyl groups, alkyloxy groups, and alkyldisulfanyl groups, wherein said alkyloxycarbonyl groups, said alkyloxy groups, and said alkyldisulfanyl groups are unsubstituted or substituted with an NR″R′″ group or a —CO—NR″R′″ group, wherein NR″R′″ is defined as above, and
(D) saturated and unsaturated 3- to 8-membered heterocyclyl groups, which are unsubstituted or substituted with at least one substituent chosen from alkyl groups and acyl groups, wherein said alkyl groups and said acyl groups are unsubstituted or substituted with an NR″R′″ group as defined above.

16. A combination of at least one group B streptogramin derivative chosen from group B streptogramin derivatives of formula (I) and salts thereof: 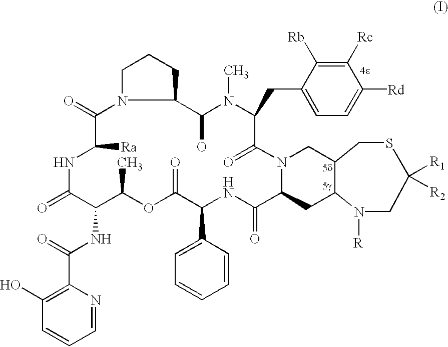
wherein:
R is chosen from a hydrogen atom, a methyl group, alkyl groups of formula R′—CH₂—, wherein R′ is chosen from straight and branched alkyl groups, and acyl groups unsubstituted or substituted with a hydroxyl group,
R₁ and R₂, which are identical or different, are each chosen from a hydrogen atom and alkyl groups,
Ra is chosen from a methyl group and an ethyl group, and
Rb, Rc and Rd are defined as follows:
1) Rb and Rc are each a hydrogen atom, and Rd is chosen from a hydrogen atom, a methylamino group, and a dimethylamino group, or
2) Rb is a hydrogen atom, Rc is chosen from a hydrogen atom, a chlorine atom, a bromine atom, and C₃-C₅ alkenyl groups, and Rd is chosen from —N(CH₃)—R′″ groups, wherein R′″ is chosen from:
(a) alkyl groups,
(b) C₂-C₄ hydroxyalkyl groups,
(c) unsubstituted C₂-C₈ alkenyl groups,
(d) C₂-C₈ alkenyl groups substituted with (i) an unsubstituted or substituted phenyl group, (ii) an unsubstituted or substituted cycloalkyl(C₃-C₆)methyl group, (iii) an unsubstituted benzyl group, (iv) a benzyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups, or (v) heterocyclylmethyl groups and heterocyclylethyl groups, wherein said heterocyclyl portions of said heterocyclylmethyl groups and said heterocyclylethyl groups are chosen from saturated and unsaturated 5-or 6-membered heterocyclyl groups comprising from 1 or 2 heteroatoms chosen from a sulphur atom, an oxygen atom, and a nitrogen atom, and wherein said heterocyclyl groups are unsubstituted or substituted with a group chosen from alkyl groups, C₂-C₈ alkenyl groups, C₃-C₆ cycloalkyl groups, saturated and unsaturated 4- to 6-membered heterocyclyl groups, an unsubstituted phenyl group, a benzyl group, or a phenyl group substituted with at least one substituent chosen from halogen atoms, a hydroxyl group, alkyl groups, alkyloxy groups, alkylthio groups, alkylsulphinyl groups, alkylsulphonyl groups, an amino group, alkylamino groups, and dialkylamino groups,
(e) a cyanomethyl group,
(f) a carboxymethyl group, and
(g) —CORe groups and —CH₂CORe groups, wherein Re is chosen from (i) —OR′e groups, wherein R′e is chosen from C₁-C₆ alkyl groups, C₂-C₆ alkenyl groups, a benzyl group, and heterocyclylmethyl groups, wherein said heterocyclyl portion is chosen from 5- or 6- membered heterocyclyl groups comprising from I or 2 heteroatoms chosen from a sulphur atom, an oxygen atom, and a nitrogen atom, (ii) alkylamino groups, (iii) alkylmethylamino groups, (iv) heterocyclylamino groups and heterocyclylmethylamino groups, wherein said heterocyclyl portion of said heterocyclylamino groups and said heterocyclylmethylamino groups is chosen from 5- or 6-membered saturated heterocyclyl groups comprising from 1 to 2 heteroatoms chosen from a sulphur atom, an oxygen atom, and a nitrogen atom, and wherein said heterocyclyl groups are unsubstituted or substituted with a group chosen from alkyl groups, a benzyl group, and alkyloxycarbonyl groups, or
3) Rb is a hydrogen atom, Rd is chosen from an —NHCH₃ group and an —N(CH₃)₂ group, and Rc is chosen from a chlorine atom and a bromine atom, and when Rd is an —N(CH₃)₂ group, Rc is chosen from C₃-C₅ alkenyl groups, or
4) Rb and Rd are each a hydrogen atom, and Rc is chosen from halogen atoms, alkylamino groups, dialkylamino groups, alkyloxy groups, a trifluoromethoxy group, thioalkyl groups, C₁-C₆ alkyl groups, and trihalomethyl groups, or
5) Rb and Rc are each a hydrogen atom, and Rd is chosen from halogen atoms, and an ethylamino group, a diethylamino group, a methylethylamino group, alkyloxy groups, a trifluoromethoxy group, alkylthio groups, alkylsulfinyl groups, alkylsulfonyl groups, C₁-C₆ alkyl groups, a phenyl group, and trihalomethyl groups, or
6) Rb is a hydrogen atom, Rc is chosen from halogen atoms, alkylamino groups, dialkylamino groups, alkyloxy groups, a trifluoromethoxy group, thioalkyl groups, and C₁-C₃ alkyl groups, and Rd is chosen from halogen atoms, an amino group, alkylamino groups, dialkylamino groups, alkyloxy groups, a trifluoromethoxy group, thioalkyl groups, C₁-C₆ alkyl groups, and trihalomethyl groups, or
7) Rc is a hydrogen atom, and Rb and Rd are each a methyl group,
unless stated otherwise, said alkyl groups are chosen from straight and branched, unsubstituted or substituted C₁-C₄ alkyl groups, and
unless stated otherwise, said acyl groups are chosen from straight and branched, unsubstituted or substituted C₁-C₄ acyl groups, and wherein said group B streptogramin is chosen from 5γ(R), 5δ(S) group B
streptogramin derivatives, salts thereof, 5γ(S), 5δ(R) group B streptogramin derivatives, salts thereof, and mixtures of any of the foregoing,
and at least one group A streptogramin derivative chosen from (A) pristinamycin IIA, pristinamycin IIB, pristinamycin IIC, pristinamycin IID, pristinamycin IIE, pristinamycin IIF, pristinamycin IIG, and salts thereof, (B) semisynthetic group A streptogramin derivatives, and salts thereof, (C) derivatives of formula (α) and salts thereof: 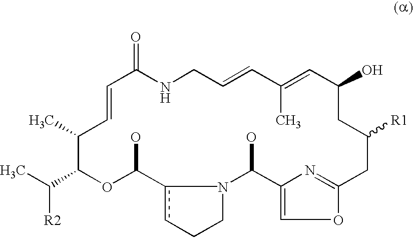
wherein:
R₁ is chosen from
(a) —NR′R″ groups, wherein R′ is chosen from a hydrogen atom and a methyl group, and R″ is chosen from a hydrogen atom, alkyl groups, cycloalkyl groups, an allyl group, a propargyl group, and a benzyl group,
(b) —OR′″ groups, wherein R′″ is chosen from a hydrogen atom, alkyl groups, cycloalkyl groups, an allyl group, a propargyl group, and a benzyl group, and
(c) —NR₃R₄ groups, wherein R₃ and R₄ are each a methyl group or form, together with the nitrogen atom to which they are attached, a saturated or unsaturated 4- or 5-membered heterocycle optionally comprising, in addition to said nitrogen atom, a hetero atom chosen from a nitrogen atom, an oxygen atom, and a sulfur atom,
R₂ is chosen from a hydrogen atom, a methyl group, and an ethyl group, and the bond --- is a single bond or a double bond, and
(D) semisynthetic derivatives of formula (β) and salts thereof: 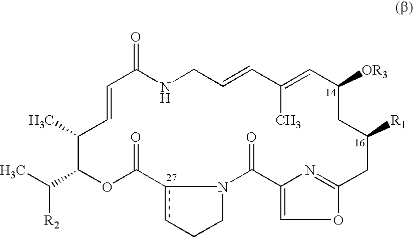
wherein:
R₁ is chosen from halogen atoms, an azido group, and a thiocyanato group,
R₂ is chosen from a hydrogen atom, a methyl group, and an ethyl group,
R₃ is chosen from a hydrogen atom and unsubstituted or substituted aliphatic ester residues, unsubstituted or substituted cycloaliphatic ester residues, unsubstituted or substituted aromatic ester residues, unsubstituted or substituted araliphatic ester residues, unsubstituted or substituted heterocyclic ester residues, and unsubstituted or substituted heterocyclylaliphatic ester residues, and
the bond --- is a single bond (27R stereochemistry) or a double bond.
